(19) United States
(12) Patent Application Publication
(10) Pub. No.: US 2026/0283730 A1
(43) Pub. Date: Sep. 24, 2026

(54) MEDICAL INSTRUMENT WRIST WITH CABLE ROUTING

(71) Applicant: Intuitive Surgical Operations, Inc., Sunnyvale, CA (US)

(72) Inventors: Thad LIEB, Santa Barbara, CA (US); Erik NELSON, Durango, CO (US)

(73) Assignee: Intuitive Surgical Operations, Inc., Sunnyvale, CA (US)

(21) Appl. No.: 19/475,533

(22) PCT Filed: Apr. 17, 2024
(86) PCT No.: PCT/US2024/024972

§ 371 Date: Oct. 15, 2025

Related U.S. Application Data
(60) Provisional application No. 63/460,401, filed on Apr. 19, 2023.

(51) Int. Cl. A61B 34/00; A61B 1/00; A61B 17/00; A61B 34/30; A61B 34/35

(52) U.S. Cl. CPC A61B 34/71; A61B 1/00009; A61B 17/00234; A61B 34/35; A61B 2017/00314; A61B 2034/302

(57) ABSTRACT

A medical device includes a proximal link, a distal link, a tool member, and at least one tension element. The proximal and distal links each include an arcuate link contact surface and an outer guide surface. The distal link rotates relative to the proximal link about a distal link rotation axis or a proximal link rotation axis throughout a rotation range. The arcuate link contact surfaces are in rolling contact throughout the rotation range. The tension element is coupled to the tool member and extends from the tool member and contacts the outer guide surfaces of the proximal and distal links. The proximal link outer guide surface is at a spaced distance from the proximal link rotation axis and the proximal link contact surface defines a radius of curvature that is substantially equal to the spaced distance of the proximal link outer guide surface from the proximal link rotation axis.

### CROSS-REFERENCE TO RELATED APPLICATIONS

This application claims priority to and the filing date benefit of U.S. Provisional Patent Application No. 63/460,401, entitled “MEDICAL INSTRUMENT WRIST WITH CABLE ROUTING,” filed Apr. 19, 2023, the disclosure of which is incorporated herein by reference in its entirety.

### BACKGROUND

The embodiments described herein relate to medical devices, and more specifically to endoscopic tools. More particularly, the embodiments described herein relate to medical devices that include wrist mechanisms with cable routing providing constant cable moment arm through range of motion of medical device.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft are inserted into a small incision or a natural orifice of a patient to position the end effector at a work site within the patient's body. The wrist mechanism can be used to change the end effector's orientation with reference to the shaft to perform the desired procedure at the work site. Known wrist mechanisms generally provide the desired mechanical degrees of freedom (DOFs) for movement of the end effector. For example, known wrist mechanisms are able to change the pitch and yaw orientation of the end effector with reference to the shaft's longitudinal axis. A wrist may optionally provide a roll DOF for the end effector with reference to the shaft, or an end effector roll DOF may be implemented by rolling the shaft, wrist, and end effector together as a unit. An end effector may optionally have additional mechanical DOFs, such as grip or knife blade motion. In some instances, wrist and end effector mechanical DOFs may be combined to provide various end effector control DOFs. For example, U.S. Pat. No. 5,792,135 (filed May 16, 1997) discloses a mechanism in which wrist and end effector grip mechanical DOFs are combined to provide an end effector yaw control DOF.

To enable the desired movement of the distal wrist mechanism and end effector, known instruments include cables that extend through the shaft of the instrument and that connect the wrist mechanism to a force transmission mechanism configured to move the cables to operate the wrist mechanism and end effector. For teleoperated systems, the force transmission mechanism is typically motor driven and is operably coupled to a computer processing system to provide a user interface for a clinical user (e.g., a surgeon) to control the instrument as a whole, as well as the instrument's components and functions.

Patients benefit from continual efforts to improve the effectiveness of MIS methods and devices. For example, reducing the size and/or the operating footprint of the shaft and wrist mechanism can allow for smaller entry incisions and reduced need for space at the surgical site, thereby reducing the negative effects of surgery, such as pain, scarring, and undesirable healing time. But producing small medical devices that implement the clinically desired functions for minimally invasive procedures can be challenging. Specifically, simply reducing the size of known wrist mechanisms by scaling down the components will not result in an effective solution because required component and material properties do not scale at relatively small physical dimensions. For example, efficient implementation of a wrist mechanism can be complicated because the cables must be carefully routed through the wrist mechanism to maintain cable tension throughout the range of motion of the wrist mechanism or end effector and to minimize the interactions (coupling effects) of motion about one rotation axis upon motion about another rotation axis. As another example, pulleys and/or contoured surfaces are generally needed to reduce cable friction, which permits operation without excessive forces being applied to the cables or other structures in the wrist mechanism. But increased localized forces that may result from smaller structures and cable bend radii (including smaller diameter cables and other wrist and end effector components) can result in undesirable lengthening (e.g., stretch or creep) of the cables during storage and use, reduced cable life, and the like.

Further, the wrist mechanism generally provides specific degrees of freedom for movement of the end effector. For example, for forceps or other grasping tools, the wrist may be able to change the end effector pitch, yaw, and grip orientations with reference to the instrument shaft. More degrees of freedom could be implemented through the wrist but would require additional actuation members (e.g., cables) in the wrist and shaft, and these additional members compete for the limited space that exists given the size restrictions required by MIS applications. Components needed to actuate other degrees of freedom, such as end effector roll or insertion/withdrawal through movement of the main tube, also compete for space at or in the shaft of the device.

A conventional architecture for a wrist mechanism in a manipulator-driven medical device uses cables pulled in and paid out by a capstan in the proximal force transmission mechanism and thereby rotate the portion of the wrist mechanism that is connected to the capstan via the cables. For example, a wrist mechanism can be operably coupled to three capstans-one each for rotations about a pitch axis, a yaw axis, and a grip axis. Each capstan can be controlled by using two cables that are attached to the capstan so that one side pays out cable while the other side pulls in an equal length of cable. With this architecture, three degrees of freedom require a total of six cables extending from the wrist mechanism proximally back along the length of the instrument's main shaft tube to the instrument's proximal force transmission mechanism. Efficient implementation of a wrist mechanism and proximal force transmission mechanism can be complicated because the cables must be carefully routed through the tool member, wrist mechanism, and proximal force transmission mechanism to maintain stability of the wrist throughout the range of motion of the wrist mechanism and to minimize the interactions (or coupling effects) of one rotation axis upon another.

With a desire to reduce the size (e.g., outside diameter) of robotic instruments, there is a need for improved wrist and cable routing designs to provide sufficient stiffness and load carrying capabilities to satisfy performance requirements with reduced size components.

Additionally, with smaller instruments, achieving the desired output force (e.g., for rotating the end effector about a pitch axis or rotating cutting blades about a grip axis) can be challenging due to the reduced space. Thus, a need also exists for wrist mechanisms with improved cable routing to optimize the output forces in several degrees of freedom.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a medical device includes a proximal wrist link, a distal wrist link, a tool member, and a tension element. The proximal wrist link includes an arcuate proximal link contact surface and a proximal link outer guide surface. The distal wrist link includes an arcuate distal link contact surface and a distal link outer guide surface. The distal link is rotatable about a distal link rotation axis and the tool member is rotatable about a tool member rotation axis. The distal wrist link rotates with reference to the proximal wrist link about the distal link rotation axis, or a proximal link rotation axis throughout a rotation range. The arcuate proximal link contact surface is in rolling contact with the arcuate distal link contact surface throughout the rotation range. The tension element is coupled to the tool member and extends from the tool member and contacts the distal link outer guide surface and the proximal link outer guide surface. Tension on the tension element urges at least one of the distal wrist link to rotate about the distal link rotation axis, the distal wrist link to rotate about the proximal link rotation axis, or the tool member to rotate about the tool member rotation axis. The proximal link outer guide surface is at a spaced distance from the proximal link rotation axis and the proximal link contact surface defines a proximal link radius of curvature, which is substantially equal to the spaced distance of the proximal link outer guide surface from the proximal link rotation axis.

In some embodiments, the medical device further includes a connector link that includes a proximal end portion and a distal end portion. The distal end portion of the connector link is coupled to the distal wrist link at the distal link rotation axis such that the distal wrist link is rotatable with reference to the connector link about the distal link rotation axis. The proximal end portion of the connector link is coupled to the proximal wrist link at the proximal link rotation axis such that the connector link is rotatable with reference to the proximal wrist link about the proximal link rotation axis.

In some embodiments, the connector link defines an internal pathway through which an elongate element can extend. In some embodiments, the elongate element is at least one of a shape fiber, an electrical wire or cable. In some embodiments, the internal pathway includes a tapered entry at a proximal end of the internal pathway. In some embodiments, a distal end of the connector link is disposed distally of the distal link rotation axis.

In some embodiments, the arcuate proximal link contact surface is at least 180 degrees and the arcuate distal link contact surface is at least 180 degrees. In some embodiments, the rotation range includes at least 45 degrees of rotation, and the proximal link radius of curvature remains constant through the rotation range. In some embodiments, the rotation range includes at least 90 degrees of rotation, and the proximal link radius of curvature extends through the rotation range.

In some embodiments, the proximal link defines an inner tension element channel. The tension element is routed from the proximal link outer guide surface to the inner tension element channel such that the tension element maintains contact with the proximal link outer guide surface throughout the rotation range. In some embodiments, the tension element is substantially tangent to an outer surface of the medical device when the tension element contacts the distal link outer guide surface and the proximal link outer guide surface.

In some embodiments, the distal link outer guide surface is at a spaced distance from the distal link rotation axis and the distal link contact surface defines a distal link radius of curvature. The distal link radius of curvature is substantially equal to the spaced distance of the distal link outer guide surface from the distal link rotation axis.

In some embodiments, the proximal wrist link defines an inner tension element channel that is positioned proximal to the proximal link outer guide surface and extends toward a centerline of the proximal wrist link. In some embodiments, the distal wrist link defines an inner tension element channel, and the tension element is routed from the inner tension element channel of the distal wrist link to the distal link outer guide surface such that the tension element maintains contact with the distal link outer guide surface throughout the rotation range. In some embodiments, the distal wrist link defines an inner tension element channel that is positioned distal to the distal link outer guide surface and extends toward a centerline of the distal wrist link.

In some embodiments, a medical device includes a proximal wrist link, a distal wrist link, a tool member, and a tension element. The proximal wrist link includes an arcuate proximal link contact surface and a proximal link outer guide surface and the distal wrist link includes an arcuate distal link contact surface and a distal link outer guide surface. The distal link is rotatable about a distal link rotation axis and the distal wrist link rotates with reference to the proximal wrist link about the distal link rotation axis or a proximal link rotation axis throughout a rotation range. The arcuate proximal link contact surface is in rolling contact with the arcuate distal link contact surface throughout the rotation range. The tension element is coupled to the tool member and extends from the tool and contacts the distal link outer guide surface and the proximal link outer guide surface. Tension on the tension element urges at least one of the distal wrist link to rotate about the distal wrist rotation axis, the proximal wrist rotation axis, or the tool member to rotate about the tool member rotation axis. The proximal link outer guide surface is at a constant distance from the proximal link rotation axis throughout the rotation range.

In some embodiments, the medical device further includes a connector link that includes a proximal end portion and a distal end portion. The distal end portion of the connector link is coupled to the distal wrist link at the distal link rotation axis such that the distal wrist link is rotatable with reference to the connector link about the distal link rotation axis. The proximal end portion of the connector link is coupled to the proximal wrist link at the proximal link rotation axis such that the connector link is rotatable with reference to the proximal wrist link about the proximal link rotation axis.

In some embodiments, the connector link defines an internal pathway through which an elongate element can extend. In some embodiments, the elongate element is at least one of a shape fiber, an electrical wire or a cable. In some embodiments, the internal pathway includes a tapered entry at a proximal end of the internal pathway. In some embodiments, a distal end of the connector link is disposed distally of the distal link rotation axis.

In some embodiments, the arcuate proximal link contact surface is at least 180 degrees and the arcuate distal link contact surface is at least 180 degrees. In some embodiments, the proximal link includes a proximal link radius of curvature, the rotation range includes at least 45 degrees of rotation, and the proximal link radius of curvature remains constant through the rotation range.

In some embodiments, the proximal link includes a proximal link radius of curvature, the rotation range includes at least 90 degrees of rotation, and the proximal link radius of curvature extends through the rotation range.

In some embodiments, the proximal link defines an inner tension element channel. The tension element is routed from the proximal link outer guide surface to the inner tension element channel such that the tension element maintains contact with the proximal link outer guide surface throughout the rotation range. In some embodiments, the tension element is substantially tangent to an outer surface of the medical device when the tension element contacts the distal link outer guide surface and the proximal link outer guide surface.

In some embodiments, the connector link defines an internal pathway through which an elongate element can extend. In some embodiments, the elongate element is at least one of a shape fiber, an electrical wire or a cable. In some embodiments, the internal pathway includes a tapered entry at a proximal end of the internal pathway. In some embodiments, a distal end of the connector link is disposed distally of the distal link rotation axis.

In some embodiments, the proximal wrist link defines an inner tension element channel that is positioned proximal to the proximal link outer guide surface and extends toward a centerline of the proximal wrist link. In some embodiments, the distal wrist link defines an inner tension element channel, and the tension element is routed from the inner tension element channel of the distal wrist link to the distal link outer guide surface such that the tension element maintains contact with the distal link outer guide surface throughout the rotation range. In some embodiments, the distal wrist link defines an inner tension element channel that is positioned distal to the distal link outer guide surface and extends toward a centerline of the distal wrist link.

Other medical instruments, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a perspective view of a manipulator unit, including a plurality of instruments, of the minimally invasive teleoperated surgery system shown in FIG. 1.

FIG. 5A is a diagrammatic top view of a distal end portion of a medical device according to an embodiment shown in a straight position.

FIG. 5B is a diagrammatic top view of the distal end portion of the medical device of FIG. 5A shown with an end effector and distal link rotated relative to a proximal link of the medical device.

FIG. 5C is a diagrammatic side view of the distal end portion of the medical device of FIG. 5A shown in a straight position.

FIG. 6 is a perspective view of a medical device according to an embodiment.

FIG. 7 is a perspective view of a distal end portion of the medical device of FIG. 6.

FIG. 8A is a top view of the distal end portion of the medical device of FIG. 7.

FIG. 8B is a side view of the distal end portion of the medical device of FIG. 7.

FIG. 9 is an exploded perspective view of the distal end portion of the medical device of FIG. 7.

FIG. 10 is a perspective view of the distal end portion of the medical device of FIG. 7 with select components removed for illustration purposes.

FIG. 11 is a top perspective view of the distal end portion of the medical device of FIG. 7 shown with the distal wrist link rotated relative to the proximal wrist link.

FIG. 12A is a top perspective view of the distal end portion of the medical device of FIG. 7 shown with the distal wrist link rotated relative to the proximal wrist link and with select components removed for illustration purposes.

FIG. 12B is a proximal perspective view of the connector link of the medical device of FIG. 7.

FIG. 13 is a perspective sectional view taken along line 13-13 in FIG. 7.

FIG. 14 is a perspective side view of the distal end portion of the medical device of FIG. 7.

FIG. 15 is a top view of the distal end portion of the medical device of FIG. 7 with portions of the medical device removed for illustration purposes.

FIG. 16 is a cross-sectional top view of a distal end portion of the medical device of FIG. 7.

FIG. 17 is a cross-sectional side view of a distal end portion of the medical device of FIG. 7.

FIG. 18 is a flowchart of a method of articulating an instrument using a wrist assembly.

FIG. 19 is a flowchart of a method of assembling a wrist assembly.

### DETAILED DESCRIPTION

The embodiments described herein can advantageously be used in a wide variety of grasping, cutting, and manipulating operations associated with minimally invasive surgery. In some embodiments, an end effector of a medical device (e.g., an instrument) can move with reference to a force transmission mechanism of the instrument in three mechanical DOFs, e.g., pitch, yaw, and roll (e.g., instrument shaft roll). There may also be one or more mechanical DOFs in the end effector itself, e.g., two jaws, each rotating with reference to a clevis (2 DOFs, which include a “grip” DOF when the jaws rotate in opposite directions in a closing manner) and a distal clevis that rotates with reference to a proximal clevis (one DOF).

The medical devices of the present application enable motion in three degrees of freedom (e.g., about a pitch axis, a yaw axis, and a grip axis) using only four tension elements (such as cables, bands, or the like), thereby reducing the total number of tension elements required for articulation, reducing the space required within the shaft and wrist for routing tension elements, reducing overall cost, and enabling further miniaturization of the wrist and shaft assemblies to promote MIS procedures. As described herein, in some embodiments, a medical device includes a wrist assembly that includes a proximal link coupled to a distal link, which is coupled to an end effector. The distal link can rotate relative to the proximal link along a rolling arcuate contact surface of each of the distal and proximal links.

The medical devices described herein can include one or more tension elements (e.g., cables, bands, or the like) that are made of any suitable material (e.g., a polymer material, or a metallic material such as tungsten) and that can be routed through wrist assembly along one or more tension element channels. To maximize the stiffness of the wrist assembly for a given tension element size (e.g., diameter) and location, the tension element contact surface radius within the wrist assembly is selected to position the outermost routing of the tension element to be tangent to the outer surface of the medical device when the wrist assembly is in a straight orientation. This creates the largest tension element moment arm that can be packaged at a given lateral offset from the centerline of the wrist assembly of the medical device. To keep the moment arm as constant as possible throughout the range of motion of the wrist assembly, when the tension element path is directed back toward the centerline of the medical device, the tension element is directed along the same radius of curvature as the tension element contact surface. To allow for this large tension element contact surface radius, the radius of curvature of the contact surfaces between the proximal and distal links of the wrist assembly are sized to be the same as the radius of curvature of the tension element guide surface. Because the tension element slides along the contact surfaces of the wrist assembly, the material of the tension elements and the links are selected to minimize wear due to friction forces. By maximizing the radius of curvature of the tension element path, the tension element bearing loads and associated wear can be reduced.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial positions and orientations. The combination of a body's position and orientation defines the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

Aspects of the invention are described with reference to a teleoperated surgical system. An example architecture of such a teleoperated surgical system is the da Vinci(®) surgical system, commercialized by Intuitive Surgical, Inc. of Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

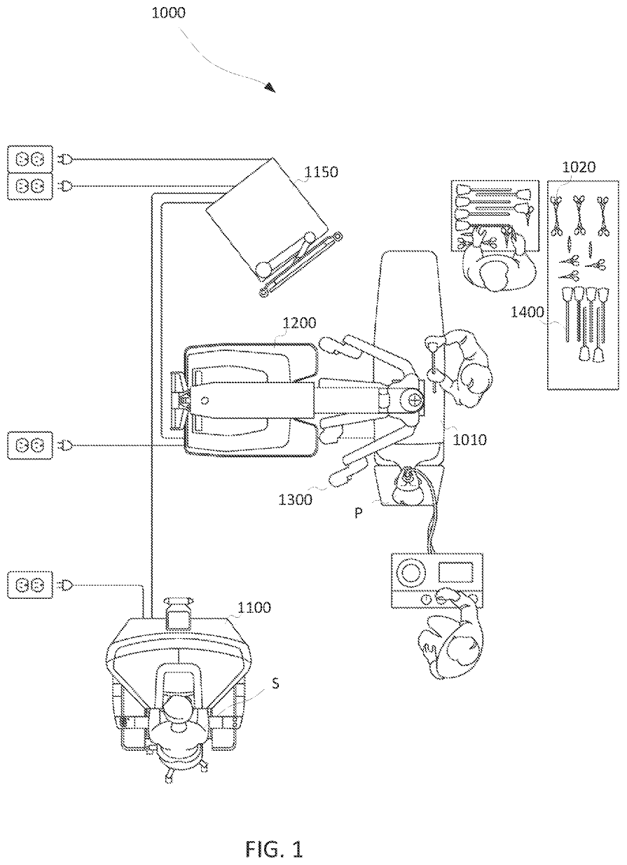

FIG. 1 is a plan view illustration of a teleoperated surgical system 1000 that operates with at least partial computer assistance (a “telesurgical system”). Both telesurgical system 1000 and its components are considered medical devices. Telesurgical system 1000 is a Minimally Invasive Robotic Surgical (“MIRS”) system used for performing a minimally invasive diagnostic or surgical procedure on a Patient P who is lying on an operating table 1010. The system can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include one or more arm assemblies 1300 that removably couple to surgical instruments 1400. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a monoscopic or stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces the instrument 1400 with another instrument 1400, for example from a tray 1020 in the operating room. Although shown as being used with the instruments 1400, any of the instruments described herein can be used with the MIRS 1000.

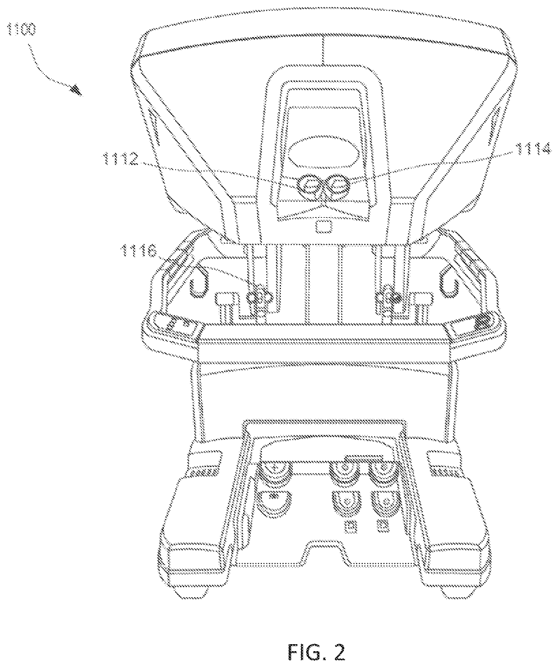

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations may be provided from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room from the patient, a completely different building from the patient, or other location remote from the patient, allowing for remote surgical procedures.

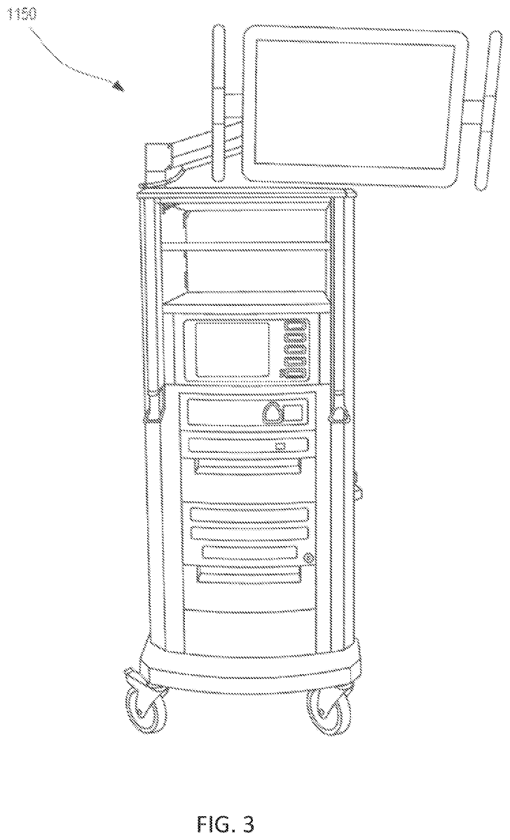

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

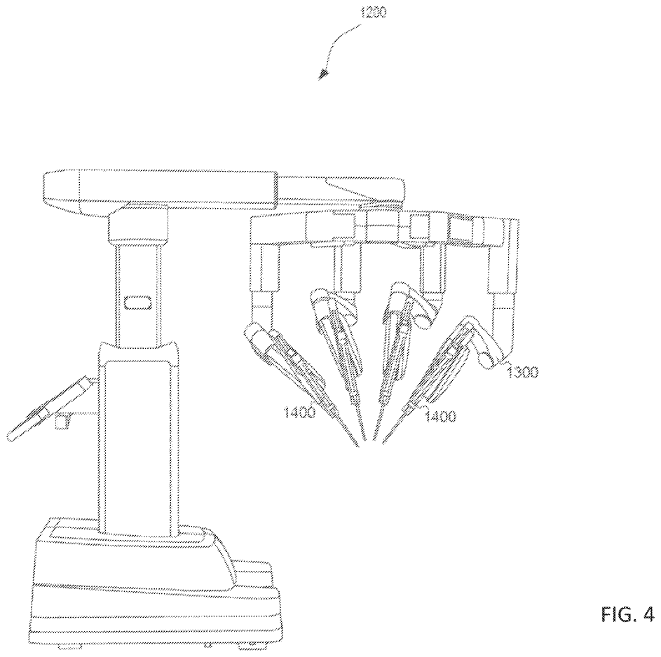

FIG. 4 shows a perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized. In various embodiments, the manipulator unit 1200 may be configured as a patient side cart with one or more arm assemblies 1300 supported and actuated by a single cart and/or individual manipulators supported and driven by separate carts. In other embodiments, the manipulator unit 1200 may be configured as a table mounted manipulator system wherein one or more arm assemblies 1300 are mounted to the operating table 1010 supporting the Patient P. In further embodiments, one or more arm assemblies 1300 may be coupled to the ceiling or other objects in the environment. It should be further appreciated that any of the arm assembly mounting configurations may be used in combination with each other.

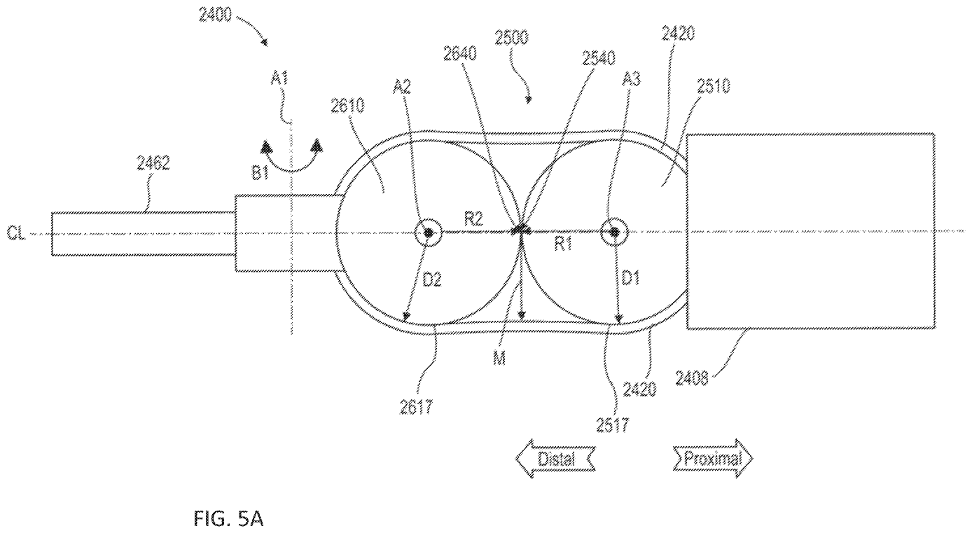

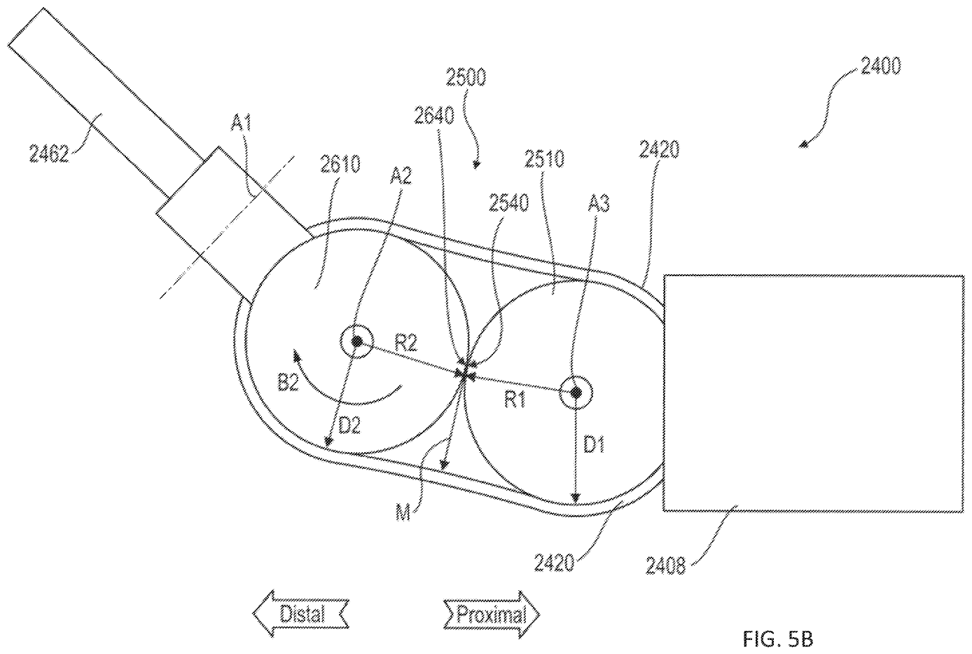

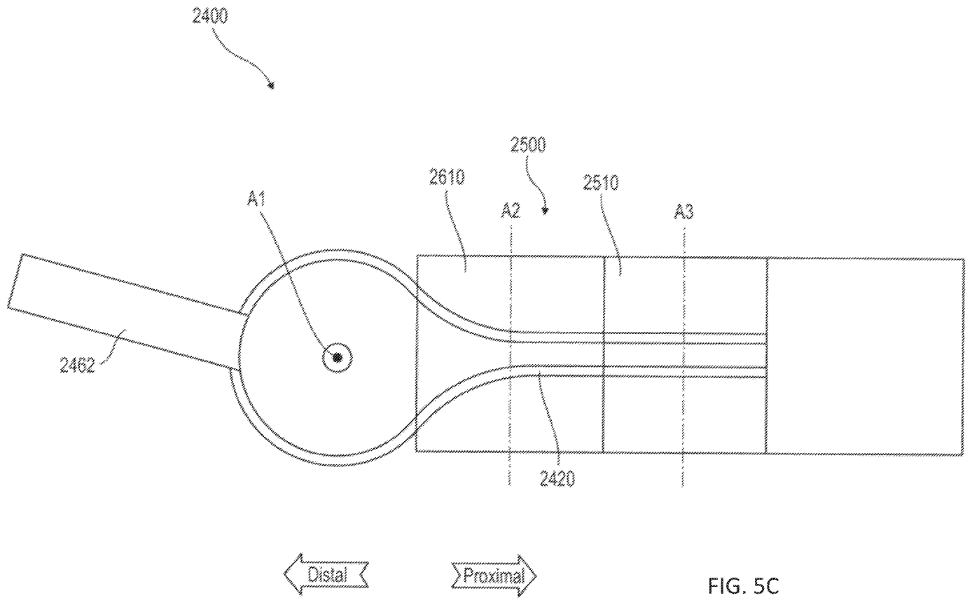

FIGS. 5A-5C are schematic illustrations of a portion of a medical device 2400 according to an embodiment. In some embodiments, the medical device 2400 or any of the components therein are optionally parts of an instrument of a surgical system that performs surgical procedures, and which surgical system can include a manipulator unit, a series of kinematic linkages, a series of cannulas, or the like. The medical device 2400 (and any of the instruments described herein) can be used in any suitable surgical system, such as the MIRS system 1000 shown and described above. The medical device 2400 includes a shaft (not shown), a proximal wrist link 2510, a distal wrist link 2610, a tool member 2462, and one or more tension elements 2420 (which can be, for example, a cable, band, or the like). Although two tension elements 2420 are shown in FIGS. 5A-5C, one or more additional tension elements 2420 can be included. The medical device 2400 is configured such that movement of the tension elements 2420 produces movement of the distal wrist link 2610, movement of the tool member 2462, or both movement of the distal wrist link 2610 and movement of the tool member 2462. In some embodiments, the tension elements 2420 can be, for example, a cable having a polymeric braided construction.

In some embodiments, the pitch of the end effector of the medical device 2400 can be changed by moving the proximal ends (not shown) of the tension elements 2420 to cause rotation of the distal wrist link 2610 as shown in FIGS. 5A and 5B. Specifically, a force transmission mechanism (not shown, but which can be similar to the force transmission mechanism 3700 described below) can pull in the proximal end of one of the tension element 2420 while also releasing the same length of the proximal end of another of the tension elements 2420 to cause the rotation of the distal wrist link 2610 as shown in FIG. 5B. In other embodiments, the medical device 2400 can include additional tension element(s) to change the yaw and grip of the end effector of the medical device 2400. For example, the yaw, grip, or both the yaw and grip can be changed by moving the proximal ends (not shown) of the tension elements 2420 as shown in FIG. 5C. Thus, the medical device 2400 can include any suitable number of tension elements to produce the desired motion of the wrist assembly 2500 and end effector 2460. For example, in some embodiments, the medical device 2400 can include two tension elements to produce the desired pitch, as described herein. In other embodiments, the medical device 2400 can include four tension elements and can operate in a manner similar to that shown and described in U.S. Patent Publication No. 2020/0390430, (filed Aug. 21, 2020), entitled “Low-Friction, Small Profile Medical Tools Having Easy-to-Assemble Components,” which is incorporated herein by reference in its entirety. In yet other embodiments, the medical device 2400 can include six tension elements and can operate in a manner similar to that shown and described in U.S. Patent Publication No. 2020/0390430 incorporated herein by reference above.

As shown in FIGS. 5A and 5B, the proximal wrist link 2510 includes an arcuate proximal link contact surface 2540 and a proximal link outer guide surface 2517. As shown, the arcuate proximal link contact surface 2540 is curved about a center that coincides with a proximal link rotation axis A3. The distal wrist link 2610 includes an arcuate distal link contact surface 2640 and a distal link outer guide surface 2617. As shown, the arcuate distal link contact surface 2640 is curved about a center that coincides with a distal link rotation axis A2. The distal wrist link 2610 can rotate relative to the proximal wrist link 2510 about the distal link rotation axis A2 and/or the proximal link rotation axis A3 throughout a rotation range such that the arcuate proximal link contact surface 2540 is in rolling contact with the arcuate distal link contact surface 2640 throughout the rotation range. In some embodiments, the arcuate proximal link contact surface 2540 and the arcuate distal link contact surface 2640 each span at least 180 degrees. Said another way, in some embodiments, either or both the angle subtended by arcuate proximal link contact surface 2540 from its center (i.e., a point that coincides with the proximal link rotation axis A3) and the angle subtended by arcuate distal link contact surface 2640 from its center (i.e., a point that coincides with the distal link rotation axis A2) can be at least 180 degrees. The proximal wrist link 2510 and the distal wrist link 2610 can be, for example, part of a wrist assembly 2500 of the medical device 2400.

As shown in FIGS. 5A and 5B, the proximal link outer guide surface 2517 is at a spaced distance D1 from the proximal link rotation axis A3 and the proximal link contact surface 2540 defines a proximal link radius of curvature R1. The proximal link radius of curvature R1 is equal to, or substantially equal to, the spaced distance D1 between the proximal link outer guide surface 2517 and the proximal link rotation axis A3. Similarly, the distal link outer guide surface 2617 is at a spaced distance D2 from the distal link rotation axis A2 and the distal link contact surface 2640 defines a distal link radius of curvature R2 that is equal to, or substantially equal to, the spaced distance D2 between the distal link outer guide surface 2617 and the distal link rotation axis R2. In some embodiments, the rotation range for rotation of the distal wrist link 2610 relative to the proximal wrist link 2510 includes at least ±45 degrees of rotation, and the proximal link radius of curvature R1 and the distal link radius of curvature R2 are constant through the rotation range. Similarly stated, in such embodiments, the proximal link radius of curvature R1 and the distal link radius of curvature R2 each form an arc having a subtended angle of at least 90 degrees. In some embodiments, the rotation range for rotation of the distal wrist link 2610 relative to the proximal wrist link 2510 includes at least ±90 degrees of rotation, and the proximal link radius of curvature R1 and distal link radius of curvature R1 are constant through the rotation range. Similarly stated, in such embodiments, the proximal link radius of curvature R1 and the distal link radius of curvature R2 each form an arc having a subtended angle of at least 180 degrees.

In various embodiments, the wrist assembly 2500 and any other wrist assemblies disclosed herein can have any suitable number of guide surfaces 2517, 2617 and corresponding tension elements 2420. For example, in some embodiments, the wrist assembly 2500 can include a proximal link outer guide surface 2517 and a distal link outer guide surface 2617 on a single side of a longitudinal centerline CL of the wrist assembly. In some embodiments, the wrist assembly 2500 can include a pair of proximal link guide surfaces 2517, with one of the guide surfaces 2517 being on one side of the longitudinal centerline CL (and providing a contact surface for a first tension element 2420) and the other guide surface 2517 being on the opposite side of the longitudinal centerline CL (and providing a contact surface for a second tension element 2420). The wrist assembly 2500 can also include a pair of distal link guide surfaces 2617, with one of the guide surfaces 2617 being on one side of the longitudinal centerline CL (and providing a contact surface for the first tension element 2420) and the other guide surface 2617 being on the opposite side of the longitudinal centerline CL (and providing a contact surface for the second tension element 2420). In addition, in some embodiments, the wrist assembly 2500 can include multiple pairs of proximal link outer guide surfaces 2517 and/or distal link outer guide surfaces 2617, for example, with multiple such guide surfaces on each side of the longitudinal centerline CL (and providing contact surfaces for a multiple tension elements 2420 on each side of the longitudinal centerline CL).

The tool member 2462 can rotate about a tool member rotation axis A1. The tool member 2462 can be, for example, a pair of jaws, a cautery device, a cutter or other medical tool. The tension element 2420 is coupled to the tool member 2462 and extends from the tool member 2462, is routed through and contacts the distal link outer guide surface 2617 and the proximal link outer guide surface 2517 and extends proximally along the medical device and is coupled to a drive component (not shown). It should be appreciated that one or more of the tension elements 2420 may be coupled to the tool member 2462, and in some embodiments, each tension element 2420 may be coupled to the tool member 2462. In some embodiments, the proximal wrist link 2510 further defines an inner tension element channel (not shown) and the tension element 2420 is routed from the proximal link outer guide surface 2517 to the inner tension element channel of the proximal wrist link 2510 such that the tension element 2420 maintains contact with the proximal link outer guide surface 2517 throughout the rotation range of the distal wrist link 2610 relative to the proximal wrist link 2510. In addition, in some embodiments, the distal wrist link 2610 further defines an inner tension element channel (not shown) and the tension element 2420 is routed from the distal link outer guide surface 2617 to the inner tension element channel of the distal link 2610 such that the tension element 2420 maintains contact with the distal link outer guide surface 2617 throughout the rotation range of the distal wrist link 2610 relative to the proximal wrist link 2510.

The distal link outer guide surface 2617 is positioned on the distal wrist link 2610 and the proximal link outer guide surface 2517 is positioned on the proximal wrist link 2510 such that the outermost routing of the tension element 2420 through the links 2510, 2610 positions the tension element 2420 to be tangent, or substantially tangent, to an outermost surface of the medical device 2400 when the distal wrist link 2610 and the proximal wrist link 2510 are in a straight configuration relative to each other, as shown in FIG. 5A. For example, the outermost surface can be an outermost surface 2408 of the wrist assembly of the medical device 2400, or an outermost surface of a shaft (not shown in FIG. 5A) of the medical device 2400. This positioning creates the largest tension element moment arm M that can be packaged at a given lateral offset from a longitudinal centerline CL of the wrist assembly of the medical device. Although the longitudinal centerline CL is shown as being linear, when the wrist assembly is moved into different orientations (i.e., when the distal wrist link 2610 rotates relative to the proximal wrist link 2510), the longitudinal centerline CL can be curved. To keep the moment arm M as constant as possible throughout the range of motion of the distal wrist link 2610, the tension element 2420 is directed along a tension element path toward the centerline CL of the medical device 2400. In this manner, the tension element 2420 is directed along the same radius of curvature of the proximal link outer guide surface 2517. To allow for this large contact surface radius for the tension element 2420, the radius of curvatures R1, R2 of the rolling arcuate contact surfaces 2540 and 2640 are sized to be the same as the radius of curvature of the proximal link outer guide surface 2517 and distal link outer guide surface 2617. Because the tension element 2420 slides along the outer guide surfaces 2517 and 2617, the materials of the tension element 2420 and the links 2510, 2610 are selected to minimize wear due to friction forces. In addition, by maximizing the radius of curvature of the guide path of the tension element 2420, the tension element bearing loads and associated wear can be reduced.

As described above, the tension element 2420 can be actuated by the drive component (not shown) such that tension on the tension element 2420 urges the distal wrist link 2610 to rotate about the distal link rotation axis A2 and/or the proximal link rotation axis A3, and/or to urge the tool member 2462 to rotate about the tool member rotation axis A1 as shown by arrow B1. As the tension element 2420 is actuated to cause the distal wrist link 2610 or the tool member 2462 to rotate, the proximal link outer guide surface 2517 remains at a constant distance D1 from the proximal link rotation axis A3 throughout the rotation range. For example, FIG. 5B illustrates the distal wrist link 2610 rotated relative to the proximal wrist link 2510 about the rotation axis A2 as shown by arrow B2. In this rotated configuration, a spaced distance D1 between the proximal link rotation axis A3 and the proximal link outer guide surface 2517 is the same as the spaced distance D1 when the distal wrist link 2610 and the proximal wrist link 2510 are in a straight configuration as shown in FIG. 5A. As also shown in FIG. 5B, the arcuate proximal link contact surface 2540 remains in contact with the arcuate distal link contact surface 2640 throughout the rotation range of the distal wrist link 2610. In addition, as described above, the moment arm M of the tension element 2420 remains substantially constant throughout the range of rotation of the wrist assembly 2500.

Although only one tool member 2462 is shown, in other embodiments, the medical device 2400 can include two or more moving tool members that cooperatively perform gripping, shearing, or cutting functions. Thus, the tool member rotation axis A1 can also function as a cutting axis as tool members rotate in opposition. Thus, in some embodiments, the medical device 2400, can provide at least three degrees of freedom (i.e., yaw motion about the tool member rotation axis A1, pitch rotation about the distal link rotation axis A2 or proximal link rotation axis A3, and a cutting motion about the tool member rotation axis A1).

In some embodiments, the wrist assembly 2500 is operatively coupled to a force transmission mechanism (not shown, but which can be similar to the force transmission mechanism 3700 described below) that functions to receive one or more motor input forces or torques (e.g., from a coupled external device such as an arm assembly 1300 from a manipulator unit 1200) and mechanically transmit the received forces or torques (e.g., via a tension element or other suitable mechanism) to move an associated one or more components in the wrist assembly 2500 (e.g., distal wrist link 2610) or tool member 2462.

In some embodiments, the medical device 2400 can also include a connector link (not shown) coupled between the distal wrist link 2610 and the proximal wrist link 2510. More specifically, the connector link includes a proximal end portion coupled to the proximal wrist link 2510 at the proximal link rotation axis A3, and a distal end portion coupled to the distal wrist link 2610 at the distal link rotation axis A2. Thus, the distal wrist link 2610 is rotatable with reference to the connector link about the distal link rotation axis A2, and the connector link is rotatable with reference to the proximal wrist link 2510 about the proximal link rotation axis A3. The distal wrist link 2610 is also rotatable about the proximal wrist link rotation axis A3 through coupling of the distal wrist link 2610 to connector link. An embodiment including a connector link is described in more detail below with reference to medical device 3400.

In some embodiments, the connector link defines an internal pathway through which an elongate element (not shown) can extend. In some embodiments, the elongate element can be, for example, a shape fiber, an electrical wire, and/or a cable. In some embodiments, the internal pathway includes a tapered entry at a proximal end of the internal pathway. In some embodiments, a distal end of the connector link is disposed distally of the distal link rotation axis A2.

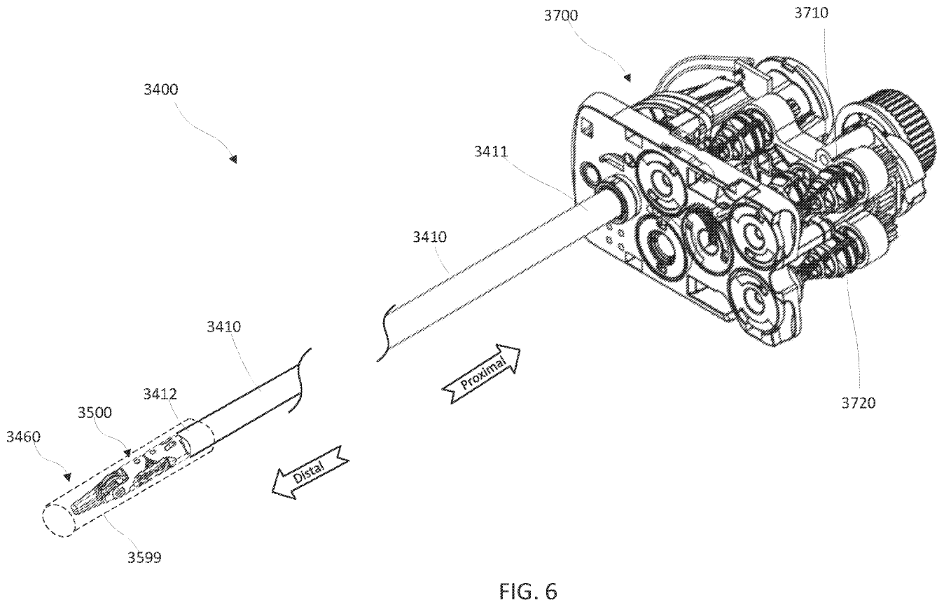

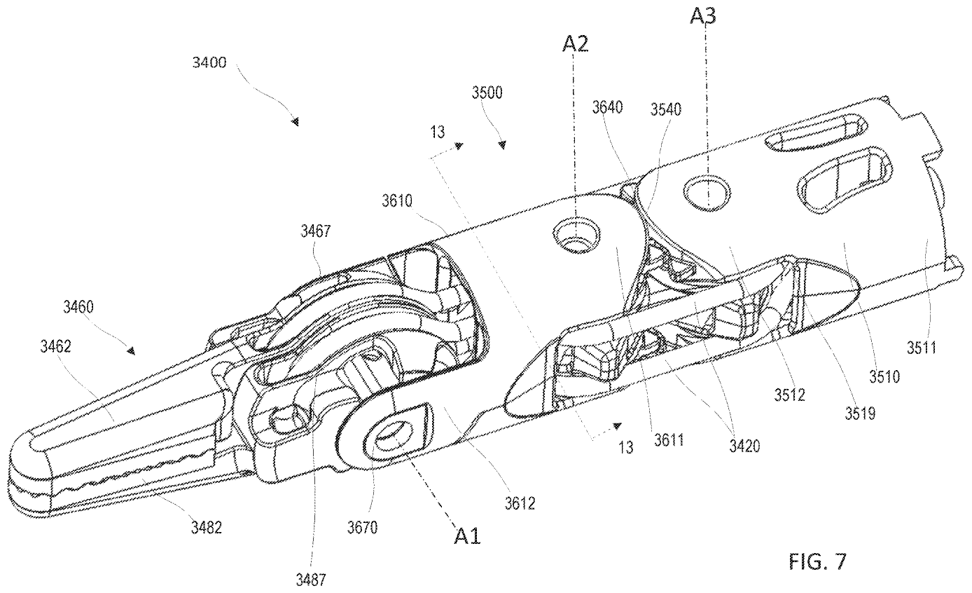

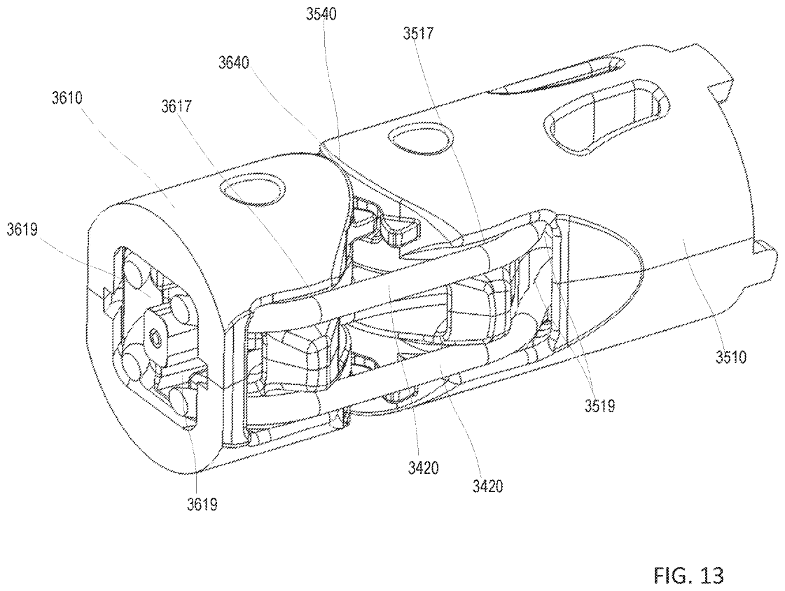

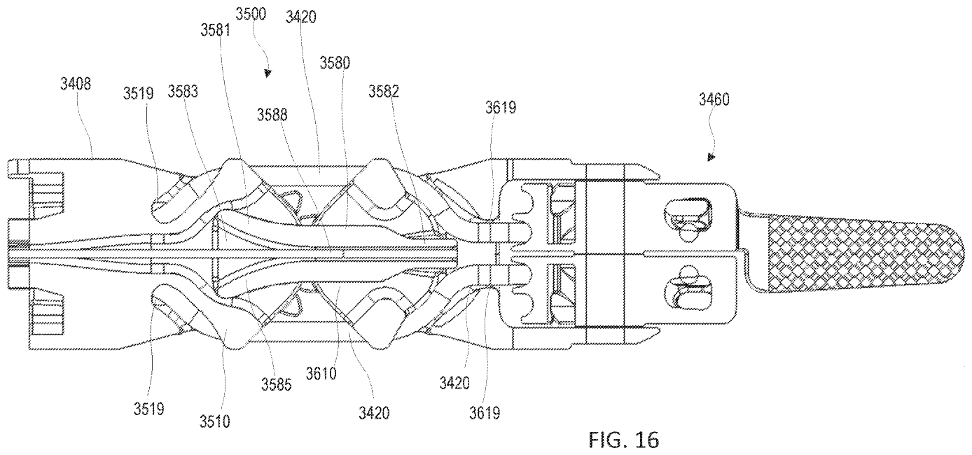

FIGS. 6-18 are various views of a medical device 3400, according to an embodiment. In some embodiments, the medical device 3400 or any of the components therein are optionally parts of an instrument for a surgical system that performs surgical procedures, and which surgical system can include a manipulator unit, a series of kinematic linkages, a series of cannulas, or the like. The medical device 3400 (and any of the instruments described herein) can be used in any suitable surgical system, such as the MIRS system 1000 shown and described above. As shown in FIG. 6, the medical device 3400 defines (or is included within) a distal boundary (or footprint) 3599 that corresponds to a cannula size or other size dictated by the surgical environment. The distal boundary 3599 can be a cylindrical shape having any suitable nominal diameter (e.g., 8 mm, 5 mm or any size therebetween). The medical device 3400 includes a force transmission mechanism 3700, a shaft 3410 (see, e.g., FIGS. 6 and 7), a distal wrist assembly 3500, a distal end effector 3460, and a set of tension elements 3420.

The medical device 3400 can include multiple tension elements 3420. For example, in some embodiments, the medical device 3400 can include two tension elements 3420 with each tension element 3420 having two segments extending along the shaft 3410 of the instrument, thereby forming four proximal end portions. Referring to FIG. 7, one of the tension elements 3420 is routed through the wrist assembly 3520 and wrapped about the pulley 3487 of the tool member 3482. This tension element 3420 has two tension element segments along the shaft 3410 with two proximal end portions that, when moved in opposite directions, can (among other things) cause rotation of the tool member 3482 about the axis A1. Another of the tension elements 3420 is routed through the wrist assembly 3520 and wrapped about the pulley 3467 of the tool member 3462. This tension element 3420 has two tension element segments along the shaft 3410 with two proximal end portions that, when moved in opposite directions, can (among other things) cause rotation of the tool member 3462 about the axis A1. This arrangement can be referred to as a “four cable” wrist and changing the pitch, yaw, or grip of the instrument 3400 can be performed by manipulating the four proximal end portions of the tension elements 3420 in a manner similar to that shown and described in U.S. Patent Publication No. 2020/0390430 incorporated herein by reference above. In other embodiments, the medical device 3400 can include four separate tension elements 3420 with two separate tension elements coupled to the pulley 3487 of the tool member 3482 and two separate tension elements coupled to the pulley 3467 of the tool member 3462, thereby creating four proximal tension element end portions. In some embodiments, the medical device 3400 can include more than two or four tension elements 3420 and more than four proximal tension element end portions. The tension elements 3420 can be, for example, cables, bands, or the like) that couple the force transmission mechanism 3700 to the distal wrist assembly 3500 and end effector 3460. In some embodiments, the tension elements 3420 can be constructed from a polymer as described above for the tension element 2420.

The medical device 3400 is configured such that movement of one or more of the tension elements 3420 produces rotation of the end effector 3460 about a first rotation axis A1 (see FIG. 7, which functions as a yaw axis, the term yaw is arbitrary), rotation of the wrist assembly 3500 about a second rotation axis A2 (also referred to as the “distal wrist rotation axis”) and/or about a third rotation axis A3 (also referred to as the “proximal wrist rotation axis”) (see FIG. 7, which functions as a pitch axis), a cutting rotation of the tool members of the end effector 3460 about the first rotation axis A1, or any combination of these movements. Changing the pitch or yaw of the medical device 3400 can be performed by manipulating the tension elements 3420 in a similar manner as that described with reference to the device 2400 described in copending International Patent Application Serial No. PCT/US2022/039942, entitled “Surgical Instrument Cable Control and Routing Structures,” the disclosure of which is incorporated herein by reference in its entirety.

The force transmission mechanism 3700 produces movement of each tension element 3420 to produce the desired movement (pitch, yaw, or grip) at the wrist assembly 3500 and the end effector 3460. Specifically, the force transmission mechanism 3700 includes components and that can be controlled to move some of the tension elements 3420 in a proximal direction (i.e., to pull in certain tension elements) while simultaneously allowing other tension elements 3420 to move in a distal direction (i.e., releasing or “paying out”). In this manner, the force transmission mechanism 3700 can cause the desired movement while also maintaining the desired tension within the tension elements 3420. As shown in FIG. 6, the proximal force transmission mechanism 3700 includes a set of drive components such as capstans 3710 and 3720 that rotate or “wind” a proximal portion of any of the tension elements 3420 to produce the desired tension element movement. In some embodiments, two proximal ends of a tension element 3420, which are associated with opposing directions of a single degree of freedom, are connected to two independent drive capstans 3710 and 3720. This arrangement, which is generally referred to as an antagonist drive system, allows for independent control of the movement of (e.g., pulling in or paying out) each of the ends of the tension elements 3420. The force transmission mechanism 3700 produces movement of the tension elements 3420, which operates to produce the desired articulation movements (pitch, yaw, or grip) at the wrist assembly 3500 and end effector 3460. Accordingly, the force transmission mechanism 3700 includes components to move a first proximal end portion of the tension element 3420 via the first capstan 3710 in a first direction (e.g., a proximal direction) and to move a second proximal end portion of the tension element 3420 via the second capstan 3720 in a second opposite direction (e.g., a distal direction). The force transmission mechanism 3700 can also move both proximal end portions of the tension element 3420 in the same direction. In this manner, the force transmission mechanism 3700 can maintain the desired tension within the tension elements 3420.

In some embodiments, the force transmission mechanism 3700 can include any of the assemblies or components described in International Patent Application Serial No. PCT/US2022/039942, entitled “Surgical Instrument Cable Control and Routing Structures,” the disclosure of which is incorporated herein by reference in its entirety. In other embodiments, however, any of the medical devices described herein can have the two ends of a tension elements wrapped about a single capstan. This alternative arrangement, which is generally referred to as a self-antagonist drive system, operates the two ends of the tension element using a single drive motor.

Moreover, although the force transmission mechanism 3700 is shown as including capstans, in other embodiments, a force transmission mechanism can include one or more linear actuators that produce translation (linear motion) of a portion of the cables. Such force transmission mechanisms can include, for example, a gimbal, a lever, or any other suitable mechanism to directly pull (or release) an end portion of any of the cables. For example, in some embodiments, the proximal force transmission mechanism 3700 can include any of the proximal force transmission mechanisms or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

The shaft 3410 can be any suitable elongated shaft that is coupled to the wrist assembly 3500 and to the force transmission mechanism 3700. Specifically, the shaft 3410 includes a proximal end 3411 that is coupled to the force transmission mechanism 3700, and a distal end 3412 that is coupled to the wrist assembly 3500 (e.g., a proximal wrist link of the wrist assembly 3500). The shaft 3410 defines a passageway or series of passageways through which the tension elements and other components (e.g., electrical wires, ground wires, or the like) can be routed from the force transmission mechanism 3700 to the wrist assembly 3500. In some embodiments, the shaft 3410 can be formed, at least in part with, for example, an electrically conductive material such as stainless steel. In such embodiments, the shaft 3410 may include an inner insulative cover or an outer insulative cover. Thus, the shaft 3410 can be a shaft assembly that includes multiple different components. For example, as shown in FIGS. 6 and 7, the shaft 3410 can include (or be coupled to) a spacer (not shown) that provides the desired fluid seals, electrical isolation features, and any other desired components for coupling the wrist assembly 3500 to the shaft 3410. Similarly stated, although the wrist assembly 3500 (and other wrist assemblies or links described herein) are described as being coupled to the shaft 3410, it is understood that any of the wrist assemblies or links described herein can be coupled to the shaft via any suitable intermediate structure, such as a spacer, a cable guide, or the like. In some embodiments, the shaft 3410 may be a substantially rigid member, while in other embodiments, the shaft 3410 may be a flexible member.

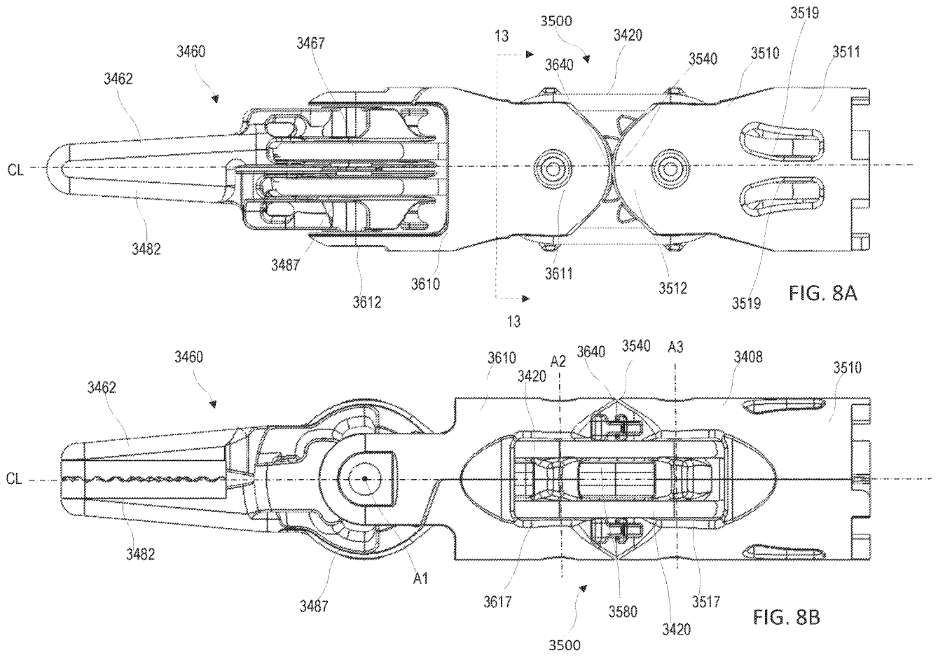

Referring to FIGS. 8B, 9, and 10, the wrist assembly 3500 includes a proximal wrist link 3510, a distal wrist link 3610 and a connector link 3580. The proximal wrist link 3510 has a proximal end portion 3511 and a distal end portion 3512. The distal wrist link 3610 has a proximal end portion 3611 and a distal end portion 3612. As described in detail herein, the connector link 3580 is coupled between the proximal wrist link 3510 and the distal wrist link 3610 to form the articulating wrist assembly 3500. The proximal wrist link 3510 is coupled to the connector link 3580 via a pinned joint such that the connector link 3580 is rotatable with reference to the proximal wrist link 3510 about the third rotation axis A3. The distal wrist link 3610 is also coupled to the connector link 3580 via a pinned joint such that the distal wrist link 3610 is rotatable with reference to the connector link 3580 about the second rotation axis A2 and is also rotatable relative to the proximal wrist link 3510 about the third rotation axis A3. In this manner, the connector link 3580 maintains the coupling between the proximal wrist link 3510 and the distal wrist link 3610 during rotation of the distal wrist link 3610 relative to the proximal wrist link 3510. The proximal wrist link 3510 is fixedly coupled to the shaft 3410 such that the proximal wrist link 3510 is not rotatable relative to the shaft. In other embodiments, however, the proximal wrist link 3510 can be rotatably coupled to the shaft 3410.

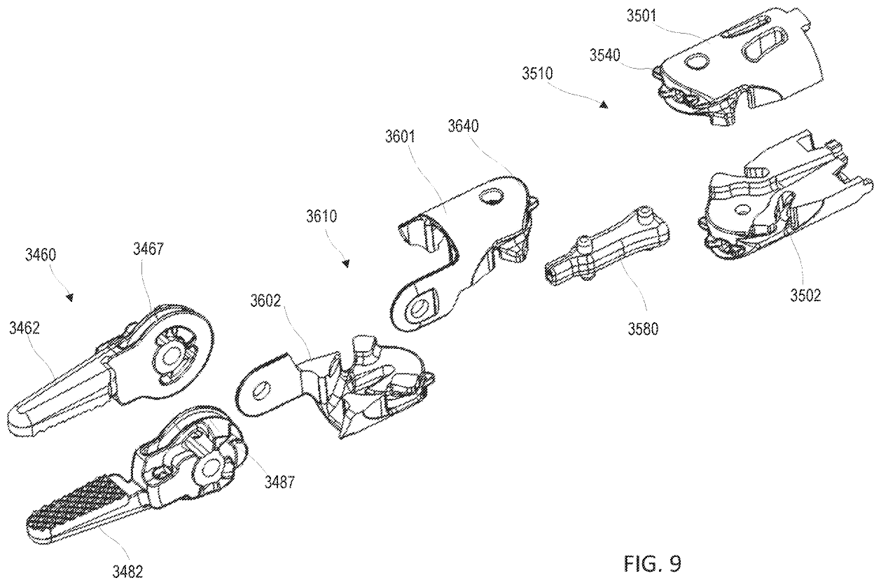

Referring to the exploded view of FIG. 9, the proximal wrist link 3510 includes a discrete first link piece 3501 and a discrete second link piece 3502. Similarly, the distal wrist link 3610 includes a discrete first link piece 3601 and a discrete second link piece 3602. The discrete pieces 3501 and 3502 are constructed as separate pieces and are later coupled together to form the proximal wrist link 3510. The discrete pieces 3601 and 3602 are constructed as separate pieces and are later coupled together to form the distal wrist link 3610. By forming the links 3510 and 3610 from two discrete pieces, the method of assembly of the medical device 3400 can be made more efficient than that for a device with a monolithically constructed wrist link. Such a wrist assembly with two-pieced links is described in more detail in U.S. Provisional Patent Application Ser. No. 63/319,971, filed Mar. 15, 2022, and entitled “Medical Device Wrist,” the disclosure of which is incorporated herein by reference in its entirety.

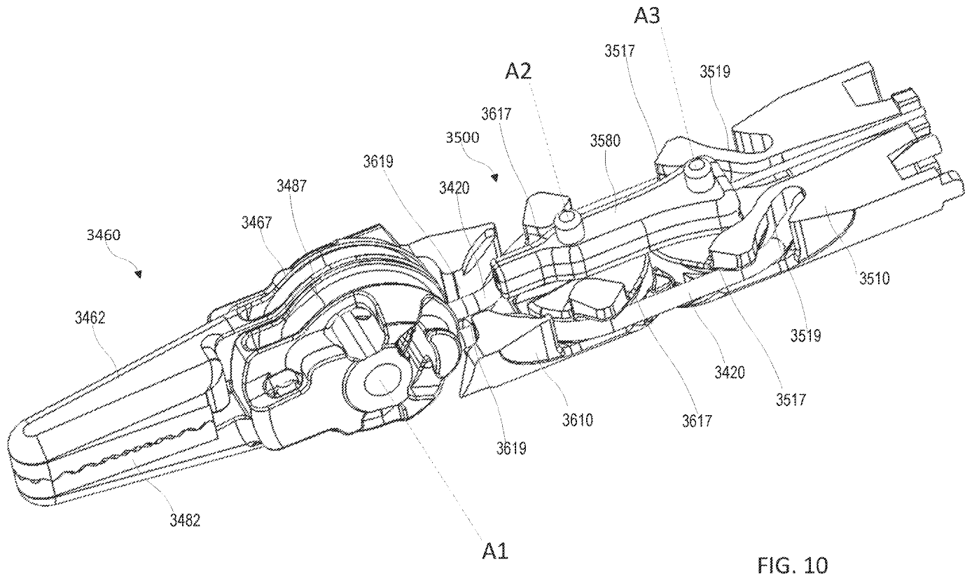

The proximal wrist link 3510 includes an arcuate proximal link contact surface 3540 and proximal link outer guide surfaces 3517 on opposite sides of the proximal wrist link 3510 (e.g., on opposite sides relative to a longitudinal centerline CL of the wrist assembly 3500 as shown in the top view of FIG. 8A). The distal wrist link 3610 includes an arcuate distal link contact surface 3640 and distal link outer guide surfaces 3617 on opposite sides of the distal wrist link 3610 (e.g., relative to the longitudinal centerline CL). Further, the outer guide surfaces 3517 and 3617 are on upper and lower portions of the links 3510, 3610 on each of the opposite sides of the links 3510, 3610. For example, the guide surfaces 3517 and 3617 are on opposite sides of the centerline CL shown in the side view of FIG. 8B. As best shown for example, in FIGS. 10, 13, 15 and 16, the proximal wrist link 3510 also includes a plurality of inner tension element channels 3519. The inner tension element channels 3519 are positioned near the longitudinal centerline CL (see e.g., FIG. 8A) of the wrist assembly 3500. Similarly, the distal wrist link 3610 also includes a plurality of inner tension element channels 3619. The inner tension element channels 3619 are also positioned near the longitudinal centerline CL of the wrist assembly 3500. Although the longitudinal centerline CL is shown as being linear, when the wrist assembly 3500 is moved into different orientations (i.e., when the link 3610 rotates relative to the link 3510), the longitudinal centerline CL can be curved.

The wrist assembly 3500 defines guide paths for the tension elements to route the tension elements 3420 through the wrist assembly 3500 and to the tool members 3462, 3482. The guide paths include the proximal link guide surfaces 3517, the distal link guide surfaces 3617 and the inner tension element channels 3519 of the proximal wrist link 3510 as described in more detail below.

The distal wrist link 3610 can rotate relative to the proximal wrist link 3510 about the distal link rotation axis A2 and/or the proximal link rotation axis A3 throughout a rotation range such that the arcuate proximal link contact surface 3540 is in rolling contact with the arcuate distal link contact surface 3640 throughout the rotation range. In some embodiments, the arcuate proximal link contact surface 3540 and the arcuate distal link contact surface 3640 each span at least 180 degrees. Said another way, in some embodiments, either or both of the angle subtended by arcuate proximal link contact surface 3540 from its center (i.e., a point that coincides with the proximal link rotation axis A3) and the angle subtended by arcuate distal link contact surface 3640 from its center (i.e., a point that coincides with the distal link rotation axis A2) can be at least 180 degrees.

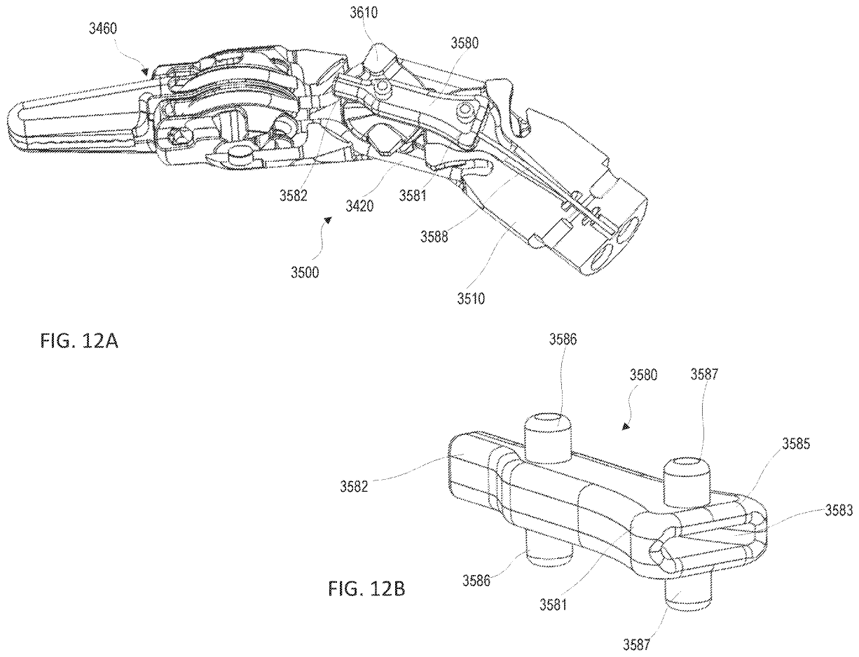

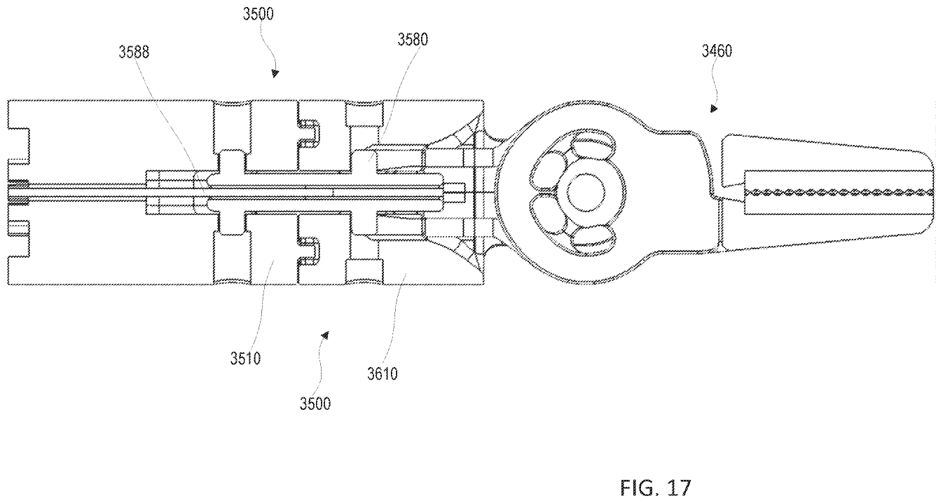

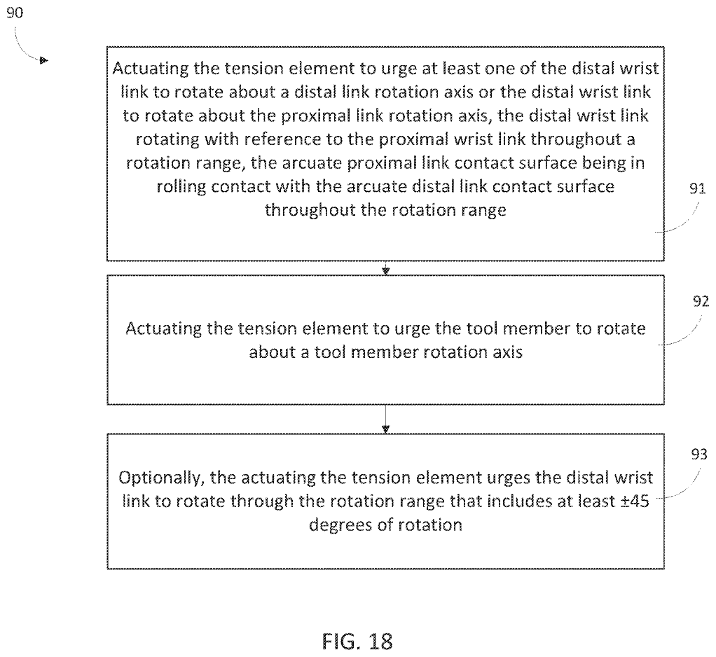

Referring to FIGS. 12A-12B, the connector link 3580 includes a proximal end 3581 and a distal end 3582 and defines an internal pathway 3583 through which one or more elongate elements 3588 (see FIGS. 17 and 18) can extend. The connector link 3580 also includes a tapered entry portion 3585 to the internal pathway 3583 at the proximal end 3581. The elongate element 3588 can be for example, an electrical wire, a cable, and/or a position sensor such as, for example, a shape fiber. In some embodiments, the internal pathway 3583 includes a tapered entry at a proximal end of the internal pathway. In some embodiments, the distal end 3582 of the connector link 3580 is disposed distally of the distal link rotation axis A2. The connector link 3580 also includes protrusions 3586 and 3587 that are used to rotatably couple the connector link 3580 to the proximal wrist link 3510 and the distal wrist link 3610. For example, the protrusions 3586 and 3587 can be received within corresponding openings (not shown) in the links 3510, 3610.

The tool members 3462 and 3482 can be, for example, a pair of jaws, a cautery device, a cutter, or other medical tool, and can rotate about the tool member rotation axis A1. The tool members 3462 and 3482 include pulleys 3467 and 3487, respectively, that can be used to drive or actuate the tool members 3462, 3482 and couple the tension elements 3420 to the tool members 3462, 3482. The tension elements 3420 are coupled to the pulleys 3467, 3487, extend through the wrist assembly 3500, through the shaft 3410 and are coupled to the drive components (e.g., capstans 3710, 3720) in the force transmission mechanism 3700. More specifically, the tension elements 3420 extend proximally from the tool members 3462, 3482, extend through and contact the distal link inner tension element channels 3619, extend through and contact the distal link outer guide surfaces 3617, extend through and contact the proximal link outer guide surfaces 3517, and are routed proximally toward the centerline CL of the wrist assembly 3500 to the inner tension element channels 3519 of the proximal wrist link 3510, and then further proximally through the shaft 3410 to the drive components (e.g., capstans 3710, 3720) in the force transmission mechanism 3700.

The proximal link inner tension element channels 3519 extend proximally of the proximal link outer guide surfaces 3517. The arcuate proximal link contact surface 3540 extends distally of the proximal link outer guide surfaces 3617 and the proximal link inner tension element channels 3519. The distal link outer guide surfaces 3617 extend proximally of the distal link inner tension element channels 3619, and the arcuate distal link contact surface 3640 extends proximally of the distal link outer guide surfaces 3617 and the distal link inner tension element channels 3619. The positioning of the inner tension element channels 3519 of the proximal wrist link 3510 near the centerline CL maintains the tension elements 3420 in contact with the proximal link outer guide surfaces 3517 throughout the rotation range of the distal wrist link 3610 relative to the proximal wrist link 3510. Similarly, the positioning of the inner tension element channels 3619 of the distal wrist link 3610 near the centerline CL maintains the tension elements 3420 in contact with the distal link outer guide surfaces 3617 throughout the rotation range of the distal wrist link 3610 relative to the proximal wrist link 3510.

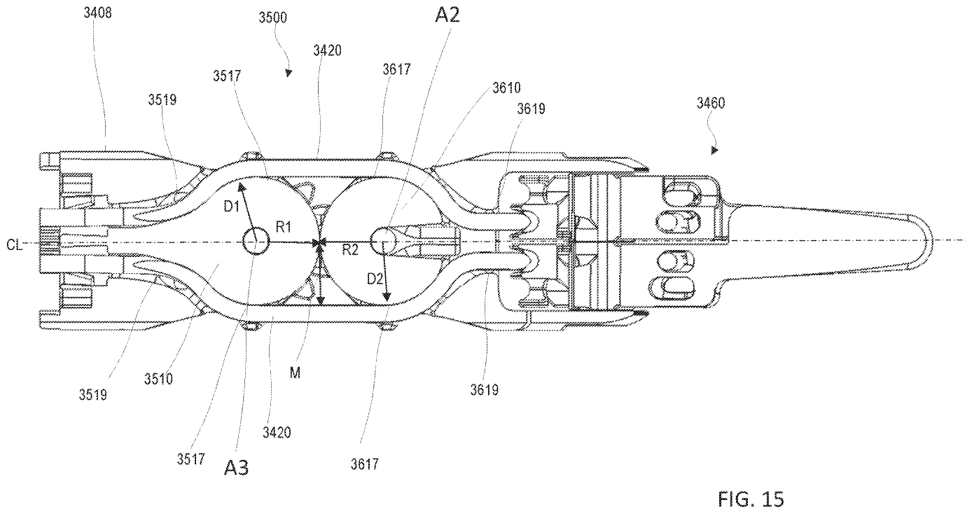

As shown in FIG. 15, the proximal link outer guide surfaces 3517 are at a spaced distance D1 from the proximal link rotation axis A3 and the proximal link contact surface 3540 defines a proximal link radius of curvature R1. The proximal link radius of curvature R1 is equal to, or substantially equal to, the spaced distance D1 between the proximal link outer guide surfaces 3517 and the proximal link rotation axis A3. Similarly, the distal link outer guide surfaces 3617 are at a spaced distance D2 from the distal link rotation axis A2 and the distal link contact surface 3640 defines a distal link radius of curvature R2. The distal link radius of curvature R2 is equal to, or substantially equal to, the spaced distance D2 between the distal link outer guide surfaces 3617 and the distal link rotation axis R2. In some embodiments, the rotation range of the distal wrist link 3610 relative to the proximal wrist link 3510 includes at least ±45 degrees of rotation, and the proximal link radius of curvature R1 and the distal link radius of curvature R2 remain constant through the rotation range. In some embodiments, the rotation range for rotation of the distal wrist link 3610 relative to the proximal wrist link 3510 includes at least ±90 degrees of rotation, and the proximal link radius of curvature R1 and distal link radius of curvature R1 extend through the rotation range.

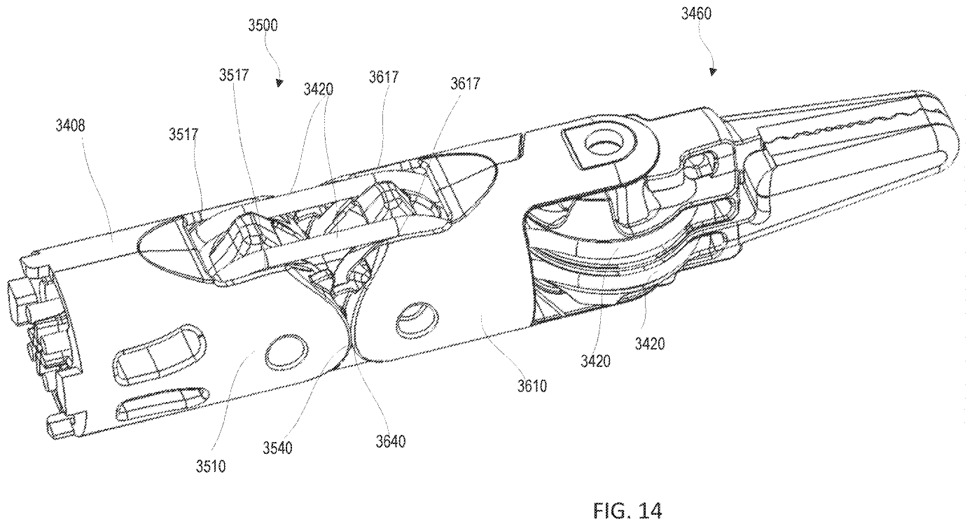

The distal link outer guide surfaces 3617 are positioned on the distal wrist link 3610 and the proximal link outer guide surfaces 3517 are positioned on the proximal wrist link 3510 such that the outermost routing of the tension elements 3420 through the links 3510, 3610 positions the tension elements 3420 to be tangent, or substantially tangent, to an outermost surface of the medical device 3400. For example, the outermost surface can be an outermost surface 3408 of the wrist assembly 3500 when the distal wrist link 3610 and the proximal wrist link 3510 are in a straight configuration relative to each other (see, e.g., FIGS. 14 and 15). In some embodiments, the outermost surface of the medical device 3400 can be the shaft 3410 (see FIG. 6) of the medical device 3400.

This positioning of the tension elements 3420 creates the largest tension element moment arm M (see FIG. 15) that can be provided at a given lateral offset from the longitudinal centerline CL of the wrist assembly 3500. To keep the moment arm M as constant as possible throughout the range of motion of the distal wrist link 3610, the tension elements 3420 are directed along a tension element path toward the centerline CL of the wrist assembly 3500. More specifically, as described above, the tension elements 3420 are directed from the proximal link outer guide surfaces 3517 and routed proximally to the proximal link inner tension element channels 3519, and toward the centerline CL of the wrist assembly 3500. The tension elements 3420 are additionally directed from the distal link outer guide surfaces 3617 and routed distally to the distal link inner tension element channels 3619 and toward the centerline of the wrist assembly 3500. In this manner, the tension elements 3420 are directed along the same radius of curvature of the proximal link outer guide surface 3517 and the same radius of curvature of the distal link outer guide surfaces 3617. To allow for this large contact surface radius for the tension elements 3420, the radius of curvatures R1, R2 of the rolling arcuate contact surfaces 3540 and 3640 are sized to be the same as the radius of curvature of the proximal link outer guide surfaces 3517 and distal link outer guide surfaces 3617. Because the tension elements 3420 slide along the outer guide surfaces 3517 and 3617, the materials of tension element 3420 and the links 3510, 3610 are preferably selected to minimize wear due to friction forces. In addition, by maximizing the radius of curvature of the guide path of the tension elements 3420, the tension elements bearing loads and associated wear can be reduced.

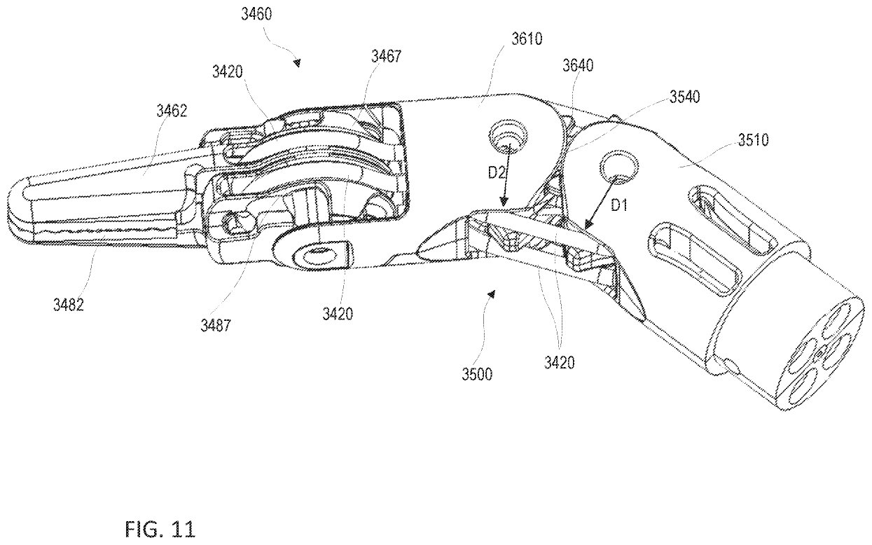

As described above, the tension elements 3420 can be actuated by the drive components (e.g., capstans 3710, 3720) such that tension on the tension elements 3420 urges the distal wrist link 3610 to rotate about the distal link rotation axis A2 and/or the proximal link rotation axis A3, and/or to urge the tool members 3462, 3482 to rotate about the tool member rotation axis A1. As the tension elements 3420 are actuated to cause the distal wrist link 3610 and/or the tool member 3462 to rotate, the proximal link outer guide surfaces 3517 remain at a constant distance D1 from the proximal link rotation axis A3 throughout the rotation range. For example, FIGS. 11 and 12A illustrate the distal wrist link 3610 in a rotated configuration relative to the proximal wrist link 3510. In this rotated configuration, the distance D1 between the proximal link rotation axis A3 and the proximal link outer guide surface 3517 is the same as the spaced distance D1 when the distal wrist link 3610 and the proximal wrist link 3510 are in a straight configuration as shown, for example, in FIG. 15. In addition, the arcuate proximal link contact surface 3540 remains in contact with the arcuate distal link contact surface 3640 throughout the rotation range of the distal wrist link 3610. As described above, the moment arm M of the tension elements 3420 remains constant throughout the range of rotation of the wrist assembly 3500.

FIG. 18 is a flowchart of a method 90 of articulating an instrument using a wrist assembly as described herein. The instrument can include, for example, a wrist assembly having a proximal wrist link and a distal wrist link, a tool member, and a tension element. The proximal wrist link includes an arcuate proximal link contact surface and a proximal link outer guide surface, and the distal wrist link includes an arcuate distal link contact surface and a distal link outer guide surface. The tension element is coupled to the tool member and extends from the tool member and contacts the distal link outer guide surface and the proximal link outer guide surface. At 91, the tension element is actuated (e.g., a first tension is applied) to urge the distal wrist link of the wrist assembly to rotate about a distal link rotation axis and/or a proximal link rotation axis throughout a rotation range. When the distal wrist link rotates with reference to the proximal wrist link about the distal link rotation axis and/or a proximal link rotation axis throughout a rotation range, the arcuate proximal link contact surface is in rolling contact with the arcuate distal link contact surface throughout the rotation range. The proximal link outer guide surface is at a spaced distance from the proximal link rotation axis and the arcuate proximal link contact surface defines a proximal link radius of curvature. The proximal link radius of curvature is substantially equal to the spaced distance of the proximal link outer guide surface from the proximal link rotation axis. In some embodiments, the proximal link outer guide surface is at a constant distance from the proximal link rotation axis throughout the rotation range. At 92, the tension element is actuated (e.g., a second tension is applied) to urge the tool member to rotate about a tool member rotation axis. In some embodiments, the tension element may be coupled to the tool member with two segments extending proximally, each segment contacting a respective distal link outer guide surface and proximal link outer guide surface. In some embodiments, the tension element may have two proximal ends (e.g., with each of the two tension element segments having a respective proximal end), and when tension is applied to one proximal end of the tension element, tension may be released on the other proximal end of the tension element. In some embodiments, the instrument can include a first tool member and a second tool member. In some embodiments, a first tension element may be coupled to the first tool member, the first tension element having two segments extending over respective distal link outer guide surfaces and proximal link outer guide surfaces, and a second tension element may be coupled to the second tool member, the second tension element having two segments extending over respective distal link outer guide surfaces and proximal link outer guide surfaces.

In some embodiments, the actuating the tension element optionally urges the distal wrist link to rotate through the rotation range that includes at least ±45 degrees of rotation, at 93. The proximal link radius of curvature remains constant through the rotation range. In some embodiments, the actuating the tension element optionally urges the distal wrist link to rotate through the rotation range that includes at least ±90 degrees of rotation.

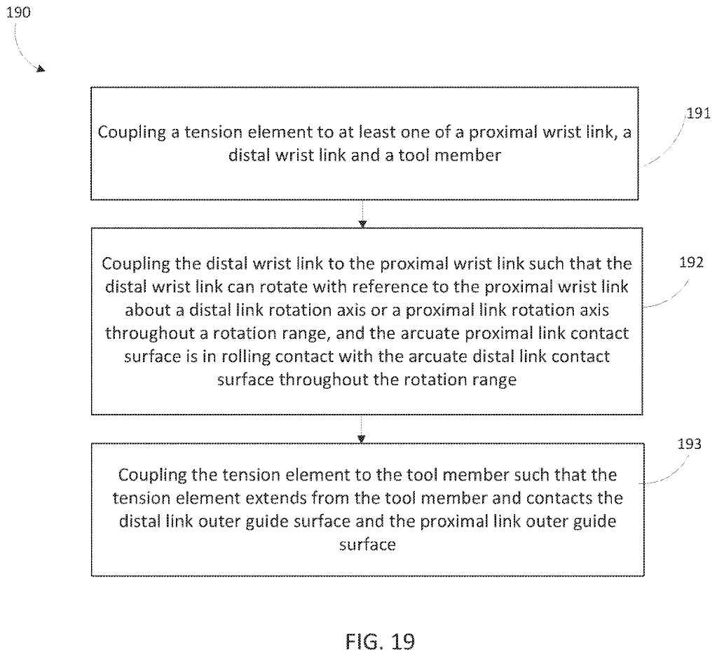

FIG. 19 is flowchart illustrating a method 190 of assembling a wrist assembly. At 191, a tension element is coupled to at least one of a proximal wrist link, a distal wrist link, and a tool member. The proximal wrist link includes an arcuate proximal link contact surface and a proximal link outer guide surface, and the distal wrist link includes an arcuate distal link contact surface and a distal link outer guide surface. The distal wrist link is rotatable about a distal link rotation axis and the tool member is rotatable about a tool member rotation axis. At 192, the distal wrist link is coupled to the proximal wrist link such that the distal wrist link can rotate with reference to the proximal wrist link about the distal link rotation axis and/or a proximal link rotation axis throughout a rotation range, and the arcuate proximal link contact surface is in rolling contact with the arcuate distal link contact surface throughout the rotation range. When the proximal wrist link is coupled to the distal wrist link, the proximal link outer guide surface is at a spaced distance from the proximal link rotation axis, and the arcuate proximal link contact surface defines a proximal link radius of curvature. The proximal link radius of curvature is substantially equal to the spaced distance of the proximal link outer guide surface from the proximal link rotation axis. At 193, the tension element is coupled to the tool member such that the tension element extends from the tool member and contacts the distal link outer guide surface and the proximal link outer guide surface. When actuated, the tension element is configured to urge at least one of the distal wrist link to rotate about the distal link rotation axis, the distal wrist link to rotate about the proximal link rotation axis, or the tool member to rotate about the tool member rotation axis. In some embodiments, the tension element may have two segments each contacting respective distal link outer guide surfaces and proximal link outer guide surfaces, with each segment being actuatable. In some embodiments, a first tension element may be coupled to a first tool member, the first tension element having two segments extending over respective distal link outer guide surfaces and proximal link outer guide surfaces, and a second tension element may be coupled to a second tool member, the second tension element having two segments extending over respective distal link outer guide surfaces and proximal link outer guide surfaces, each of the tension element segments being actuatable.

While various embodiments have been described above, it should be understood that the embodiments have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body, or the like.

For example, any of the components of a surgical instrument described herein can be constructed from any material, such as medical grade stainless steel, nickel alloys, titanium alloys, or the like. Further, any of the links, tool members, tension elements, or components described herein can be constructed from multiple pieces that are later joined together. For example, in some embodiments, a link can be constructed by joining together separately constructed components. In other embodiments however, any of the links, tool members, tension elements, or components described herein can be monolithically constructed.

In some embodiments, any of the tension elements described herein (including the tension elements 2420, 3420) can be a cable having any suitable material (e.g., a polymer material, or a metallic material such as tungsten). In some embodiments, a distal end portion of any of the tension elements described herein can include an oil coating. In some embodiments, a distal end portion of any of the tension elements described here can include a hydrophobic material. In some embodiments, any of the tension elements described herein (including the tension elements 2420, 3420) can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the tension element to a capstan within a proximal force transmission mechanism (e.g., the force transmission mechanism 3700) and/or an end effector. Such ability can also improve slip characteristics (e.g., help prevent the cable from slipping) during operation of the medical device. Such materials may or may not need a coating or other surface treatment to increase the frictional characteristics.

Although the instruments are generally shown as having an axis of rotation of the tool members (e.g., axis A1) that is normal to an axis of rotation of the wrist members (e.g., axis A2, axis A3), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device, comprising:
a proximal wrist link, a distal wrist link, a tool member, and a tension element;
wherein the proximal wrist link includes an arcuate proximal link contact surface and a proximal link outer guide surface;
wherein the distal wrist link includes an arcuate distal link contact surface and a distal link outer guide surface;
wherein the distal wrist link is rotatable about a distal link rotation axis and the tool member is rotatable about a tool member rotation axis;
wherein the distal wrist link rotates with reference to the proximal wrist link about the distal link rotation axis or a proximal link rotation axis throughout a rotation range, the arcuate proximal link contact surface being in rolling contact with the arcuate distal link contact surface throughout the rotation range;
wherein the tension element is coupled to the tool member and extends from the tool member and contacts the distal link outer guide surface and the proximal link outer guide surface;
wherein tension on the tension element urges at least one of the distal wrist link to rotate about the distal link rotation axis, the distal wrist link to rotate about the proximal link rotation axis, or the tool member to rotate about the tool member rotation axis;
wherein the proximal link outer guide surface is at a spaced distance from the proximal link rotation axis; and
wherein the arcuate proximal link contact surface defines a proximal link radius of curvature, the proximal link radius of curvature being substantially equal to the spaced distance of the proximal link outer guide surface from the proximal link rotation axis.

2. The medical device of claim 1, further comprising:
a connector link that includes a proximal end portion and a distal end portion, wherein the distal end portion of the connector link is coupled to the distal wrist link at the distal link rotation axis such that the distal wrist link is rotatable with reference to the connector link about the distal link rotation axis, and the proximal end portion of the connector link is coupled to the proximal wrist link at the proximal link rotation axis such that the connector link is rotatable with reference to the proximal wrist link about the proximal link rotation axis.

3. The medical device of claim 1, wherein:
the arcuate proximal link contact surface comprises at least 180 degrees; and
the arcuate distal link contact surface comprises at least 180 degrees.

4. The medical device of claim 1, wherein:
the rotation range includes at least ±45 degrees of rotation, and the proximal link radius of curvature remains constant through the rotation range.

5. The medical device of claim 1, wherein:
the rotation range includes at least ±90 degrees of rotation, and the proximal link radius of curvature extends through the rotation range.

6. The medical device of claim 1, wherein:
the proximal wrist link defines an inner tension element channel, the tension element is routed from the proximal link outer guide surface to the inner tension element channel such that the tension element maintains contact with the proximal link outer guide surface throughout the rotation range.

7. The medical device of claim 1, wherein:
the tension element is substantially tangent to an outer surface of the medical device when the tension element contacts the distal link outer guide surface and the proximal link outer guide surface.

8. The medical device of claim 1, wherein:
the distal link outer guide surface is at a spaced distance from the distal link rotation axis; and
the distal link contact surface defines a distal link radius of curvature, the distal link radius of curvature being substantially equal to the spaced distance of the distal link outer guide surface from the distal link rotation axis.

9-12. (canceled)

13. The medical device of claim 1, wherein:
the proximal wrist link defines an inner tension element channel, the inner tension element channel is proximal to the proximal link outer guide surface and extends toward a centerline of the proximal wrist link.

14. The medical device of claim 1, wherein:
the distal wrist link defines an inner tension element channel, the tension element is routed from the inner tension element channel of the distal wrist link to the distal link outer guide surface such that the tension element maintains contact with the distal link outer guide surface throughout the rotation range.

15. The medical device of claim 1, wherein:
the distal wrist link defines an inner tension element channel, the inner tension element channel is distal to the distal link outer guide surface and extends toward a centerline of the distal wrist link.

16. A medical device, comprising:
a proximal wrist link, a distal wrist link, a tool member, and a tension element;
wherein the proximal wrist link includes an arcuate proximal link contact surface and a proximal link outer guide surface;
wherein the distal wrist link includes an arcuate distal link contact surface and a distal link outer guide surface;
wherein the distal wrist link is rotatable about a distal link rotation axis;
wherein the distal wrist link rotates with reference to the proximal wrist link about the distal link rotation axis or a proximal link rotation axis throughout a rotation range, the arcuate proximal link contact surface being in rolling contact with the arcuate distal link contact surface throughout the rotation range;
wherein the tension element is coupled to the tool member and extends from the tool member and contacts the distal link outer guide surface and the proximal link outer guide surface;
wherein tension on the tension element urges at least one of the distal wrist link to rotate about the distal link rotation axis, the proximal link rotation axis, or the tool member to rotate about a tool member rotation axis;
wherein the proximal link outer guide surface is at a constant distance from the proximal link rotation axis throughout the rotation range.

17. The medical device of claim 16, further comprising:
a connector link that includes a proximal end portion and a distal end portion, wherein the distal end portion of the connector link is coupled to the distal wrist link at the distal link rotation axis such that the distal wrist link is rotatable with reference to the connector link about the distal link rotation axis, and the proximal end portion of the connector link is coupled to the proximal wrist link at the proximal link rotation axis such that the connector link is rotatable with reference to the proximal wrist link about the proximal link rotation axis.

18. The medical device of claim 16, wherein:
the arcuate proximal link contact surface comprises at least 180 degrees; and
the arcuate distal link contact surface comprises at least 180 degrees.

19. The medical device of claim 16, wherein:
the proximal wrist link includes a proximal link radius of curvature;
the rotation range includes at least ±45 degrees of rotation, and the proximal link radius of curvature remains constant through the rotation range.

20. The medical device of claim 16, wherein:
the proximal wrist link includes a proximal link radius of curvature;
the rotation range includes at least ±90 degrees of rotation, and the proximal link radius of curvature extends through the rotation range.

21. The medical device of claim 16, wherein:
the proximal wrist link defines an inner tension element channel, the tension element is routed from the proximal link outer guide surface to the inner tension element channel such that the tension element maintains contact with the proximal link outer guide surface throughout the rotation range.

22. The medical device of claim 16, wherein:
the tension element is substantially tangent to an outer surface of the medical device when the tension element contacts the distal link outer guide surface and the proximal link outer guide surface.

23-27. (canceled)

28. The medical device of claim 16, wherein:
the distal wrist link defines an inner tension element channel, the tension element is routed from the inner tension element channel of the distal wrist link to the distal link outer guide surface such that the tension element maintains contact with the distal link outer guide surface throughout the rotation range.

29. The medical device of claim 16, wherein:
the distal wrist link defines an inner tension element channel, the inner tension element channel is distal to the distal link outer guide surface and extends toward a centerline of the distal wrist link.

30. (canceled)
